(19) United States
(12) Patent Application Publication
(10) Pub. No.: US 2023/0024285 A1
(43) Pub. Date: Jan. 26, 2023

(54) APPARATUS, SYSTEM AND METHOD FOR MONITORING PERSONS INCLUDING WHILE SLEEPING

(71) Applicant: Smartsnugg IP Pty Ltd, Victoria (AU)

(72) Inventors: Matthew HEARSCH, Victoria (AU); Simon FRENCH, Victoria (AU)

(21) Appl. No.: 17/788,722

(22) PCT Filed: Dec. 24, 2020
(86) PCT No.: PCT/AU2020/051436

§ 371 Date: Jun. 23, 2022

(30) Foreign Application Priority Data
Dec. 24, 2019 (AU) 2019904943

(51) Int. Cl. A61B 5/00; A61B 5/01; A61B 5/11; A41B 13/06; A41D 13/12

(52) U.S. Cl. CPC A61B 5/6804; A61B 5/01; A61B 5/11; A61B 5/746; A41B 13/065; A41D 13/1236; A61B 2562/0271; A61B 2562/18; A61B 2562/0219; A61B 2560/0412; A61B 2503/06; A61B 2503/04

(57) ABSTRACT

An apparatus for monitoring a person, including while sleeping, the apparatus including:
a. a garment for a person to wear while sleeping;
b. a rigid sensor device configured to detect a temperature and/or a motion of the rigid sensor device while the person is wearing the garment; and
c. an elastic pocket configured to hold the rigid sensor device tightly inside the garment during the detection of the temperature and/or the motion.

### TECHNICAL FIELD

The present disclosure relates to an apparatus, system and method for monitoring persons, including garments and bedding for persons while sleeping, particularly children and infants.

### BACKGROUND

Existing systems for monitoring persons, particularly children (including infants) while they sleep may lack desirable functionality, e.g., they may be insufficiently accurate (e.g., when attempting to measure temperature or movement of the person) when attached to loose sleeping garments, such as infant sleeping bags and pyjamas, or they may be uncomfortable to wear (e.g., due to tight straps or hard protuberances), or they may be inconvenient to assemble or arrange on the children, or they may be difficult to keep clean, or they may require constant recharging, or they may be designed for daily removal.

It is desired to address one or more deficiencies in existing systems for monitoring persons while they sleep, or to at least provide a useful alternative.

### SUMMARY

Disclosed herein is an apparatus for monitoring a person, including while sleeping, the apparatus including: a garment for a person to wear while sleeping; a rigid sensor device configured to detect a temperature and/or a motion of the rigid sensor device while the person is wearing the garment; and an elastic pocket configured to hold the rigid sensor device tightly inside the garment during the detection of the temperature and/or the motion.

The rigid sensor device includes: a housing; and an electronic system in the housing.

The electronic system includes electronic sensor components configured to detect the temperature and/or the motion of the garment, including at least one electronic direction sensor. The rigid sensor device is configured to detect the motion of the garment by detecting a flip of the garment using the at least one electronic direction sensor.

The at least one electronic direction sensor includes at least one of an accelerometer and a gyroscope.

The electronic system includes any one or more of: an internal power source that provides power in the electronic system; a temperature sensor module configured to sense the temperature; a timer module configured to generate a WAKE signal; an accelerometer module configured to measure the acceleration of the rigid sensor device; a gyroscope module configured to measure rotation of the rigid sensor device; a wireless module configured to receive signals from the temperature sensor module representing the measured temperature, and/or from the accelerometer module representing the measured the acceleration; and an antenna configured to communicate wirelessly.

The housing is formed of at least one rigid material including a thermal conductor or a thermally conductive portion, and the rigid sensor device is configured to detect the temperature of the garment by electronically monitoring a temperature of the thermal conductor or thermally conductive portion.

The elastic pocket includes an openable closure with an elastic throat through which the rigid sensor device can be inserted and removed from the elastic pocket, wherein the rigid sensor device has a maximum width section, in a direction perpendicular to a long axis of the rigid sensor device, with a width greater than the elastic throat, such that the elastic throat resists the insertion and the removal of the rigid sensor device into and from the pocket. Optionally the insertion and the removal of the rigid sensor device requires a force between 5 and 15 N.

The elastic pocket includes an elastic pouch. The rigid sensor device and the elastic pouch are both flat so the rigid sensor device is held flat in the elastic pocket, and the elastic pouch is longer than it is wide so a longer dimension of the rigid sensor device can align with a longer dimension of the elastic pouch to resist flat rotation of the rigid sensor device.

The elastic pocket includes an inner-facing fabric, corresponding to an inner-facing side of the rigid sensor device, that is thermally permissive relative to a body fabric of the garment to mitigate thermal differences between air inside the garment and the inner-facing side of the rigid sensor device.

The rigid sensor device includes a housing body, a battery door, a water-resistant join and/or a water-resistant seal.

Disclosed herein is a method for making an apparatus, the method including: providing a garment; attaching an elastic pocket to the garment for tightly holding a rigid sensor device inside the garment while a person is wearing the garment; and configuring the rigid sensor device to detect a temperature and/or a motion of the rigid sensor device.

The method includes any one or more of: assembling electronic sensor components to form an electronic system of the rigid sensor device; forming a housing body of the rigid sensor device, including two portions and a battery door, and optionally forming the housing body with a water-resistant join and/or a water-resistant seal; forming a thermal conductor of the rigid sensor device; inserting a battery into a battery holder of the rigid sensor device; and sewing and/or bonding the elastic pocket into the garment.

Disclosed herein is a method for monitoring a person, including while sleeping, the method including: holding a rigid sensor device, by an elastic pocket, tightly inside a garment to resist movement of the rigid sensor device relative to the garment; and electronically monitoring a temperature and/or a motion of the rigid sensor device while a person is wearing the garment.

The method includes: housing an electronic system for the electrical monitoring in a water-resistant housing.

The method includes detecting a flip of the garment by: electronically monitoring for a change in orientation of the rigid sensor device by one or more electronic direction sensors measuring an axis; detecting the change in orientation of the rigid sensor device when the axis changes direction more than a selected threshold (“flip threshold”); and generating a flip alert or alarm signal when the direction change (or “flip”) is detected.

The method includes any one or more of: powering the rigid sensor device with an internal power source; sensing the temperature using a temperature sensor module of the rigid sensor device; generating a WAKE signal to transmit communication signals; measuring the motion using an accelerometer module and/or a gyroscope module of the rigid sensor device; transmitting wireless communication signals using a wireless module and an antenna of the rigid sensor device; and electronically monitoring a temperature of a thermal conductor or a thermally conductive portion of the rigid sensor device.

The method includes any one or more of: manually inserting the rigid sensor device into the elastic pocket, optionally including holding open a flap of the elastic pocket; and manually removing the rigid sensor device from the elastic pocket.

The method includes pushing the rigid sensor device through an elastic throat of the elastic pocket in a first orientation, optionally with a force between 5 and 15 N; and rotating the rigid sensor device when in the elastic pocket from the first orientation to a second orientation that is perpendicular to the first orientation.

The method includes: the elastic pocket holding the rigid sensor device flat relative to an inner face of the garment; and the elastic pocket resisting rotation of the rigid sensor device in the elastic pocket.

The method includes: mitigating a thermal difference between air inside the garment and an inner-facing side of the rigid sensor device relative to a thermal differences between air inside the garment and air outside the garment.

### BRIEF DESCRIPTION OF THE DRAWINGS

Embodiments are described hereinafter with reference to the accompanying drawings in which:

FIG. 1 is a photograph of an apparatus for monitoring a person, including while sleeping;

FIG. 2 is a front perspective schematic view of an elastic pocket for holding a rigid sensor device;

FIG. 3 is a rear schematic view of the elastic pocket;

FIG. 4 is a front schematic view of the elastic pocket in an open condition;

FIG. 5 is a front schematic view of the rigid sensor device;

FIG. 6 is a plan schematic view of the elastic pocket in the open condition;

FIG. 7 is plan schematic view of the rigid sensor device;

FIG. 8 is a further front perspective schematic view of the rigid sensor device;

FIG. 9 is a further front perspective schematic view of the rigid sensor device;

FIG. 10 is a rear perspective schematic view of the rigid sensor device;

FIG. 11 is a partially exploded rear schematic view of the rigid sensor device;

FIG. 12 is a rear schematic view of a front portion of the rigid sensor device;

FIG. 13 is a side schematic view of the rigid sensor device;

FIG. 14A is a front exploded schematic view of the rigid sensor device;

FIG. 14B is a rear exploded schematic view of the rigid sensor device;

FIG. 15 is a circuit diagram of an electronic system in the rigid sensor device;

FIG. 16 is a firmware architecture of the electronic system; and

FIG. 17 is an architecture of a monitoring system including the apparatus.

### DETAILED DESCRIPTION

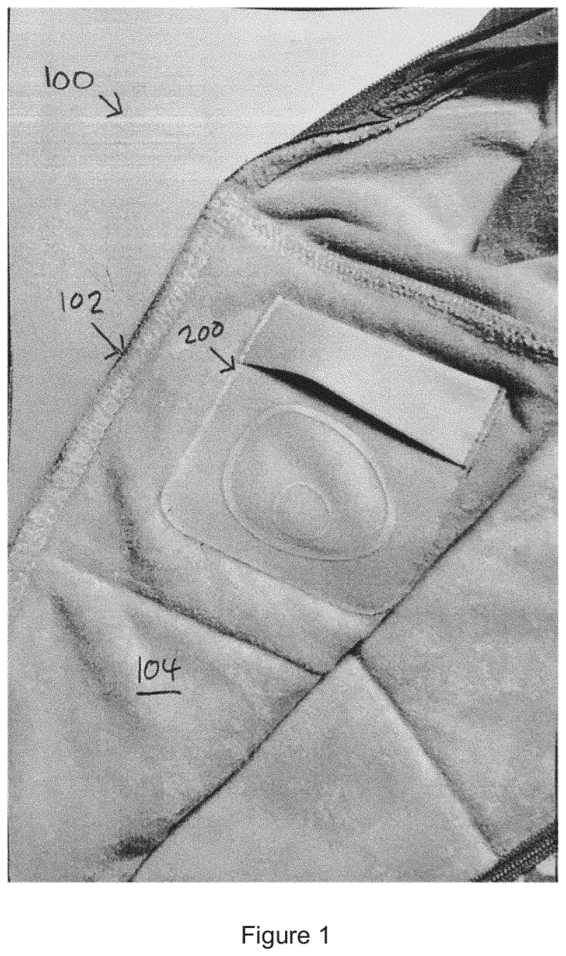

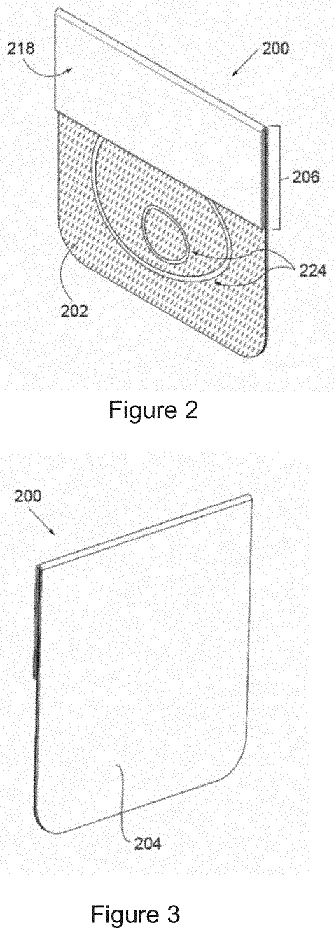

Described herein, and shown in FIGS. 1 and 5, is an apparatus 100 for monitoring a person, including while sleeping, including:
a. a garment 102 (which can be referred to as a “sleeping garment” hereinafter) for a person to wear while sleeping;
b. a rigid sensor device 300 configured to detect a temperature and/or a motion of the rigid sensor device 300 in the sleeping garment 102 while the person is wearing the sleeping garment 102; and
c. an elastic pocket 200 configured to hold the rigid sensor device 300 tightly inside the sleeping garment 102 during the detection of the temperature and/or the motion.

Detection of the motion can include detection of orientation of the rigid sensor device 300, and/or detection of motion of the rigid sensor device 300. The motion refers to position changing with time, represented by a time series of position values (e.g., based on acceleration values and/or rotation values), which can be zero if there is no change in position.

The apparatus 100 may be referred to as an “assembly” because it is assembled from a plurality of parts. The person can be a child, and the term “child”, as used herein, includes an infant.

The sleeping garment 102 is relatively tight fitting on the person so that rigid sensor device 300 remains relatively fixed to the person during use to mitigate erroneous temperature and/or motion signals. The sleeping garment 102 may include elastic material at least in portions to fit the person tightly.

The sleeping garment 102 (which may be referred to as “sleeping equipment” or “bed clothes”) can be:
a. a sleeping bag (which may be referred to as a “sleep bag”, a “swaddle” or a “sleep suit”) for children or infants; or
b. a pyjama garment for children or infants, the pyjama garment including a pyjama top and optionally a pyjama bottom.

The pyjama top may be a singlet top, a short-sleeved top, or a long-sleeved top. The pyjama top and the pyjama bottom may comprise a sleeping suit including feet (which may be referred to as a “onesie”) for children or infants to wear while sleeping.

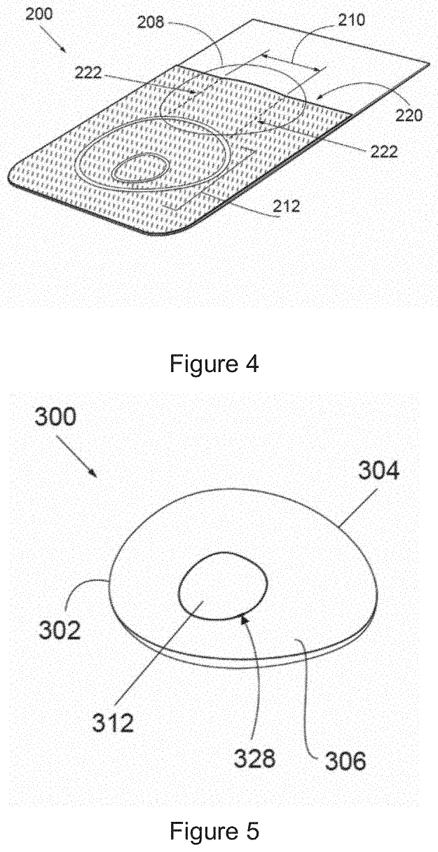

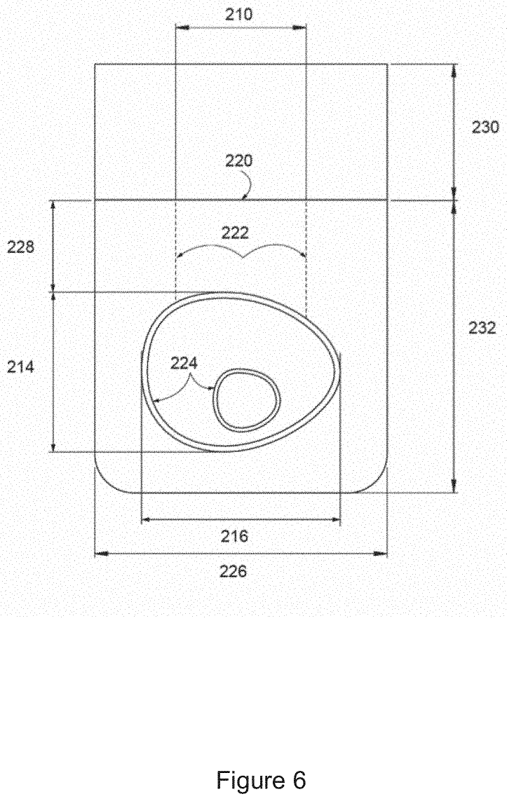

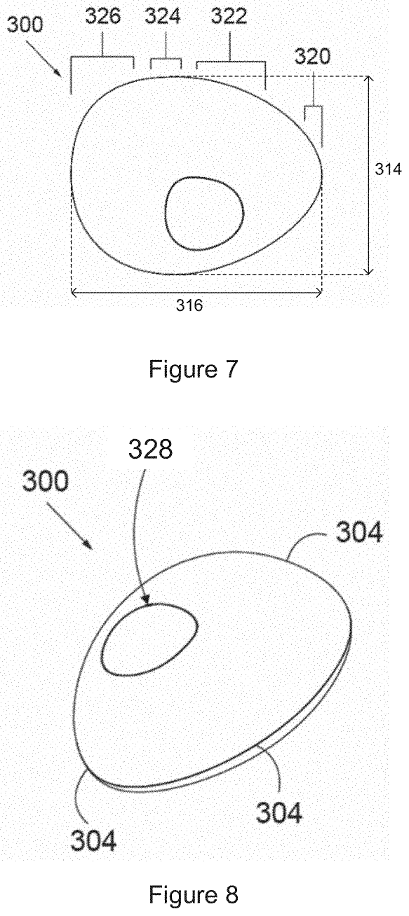

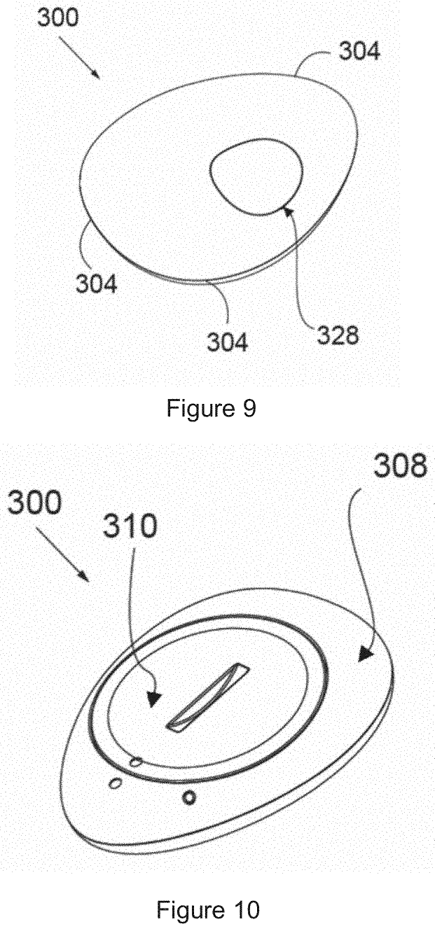

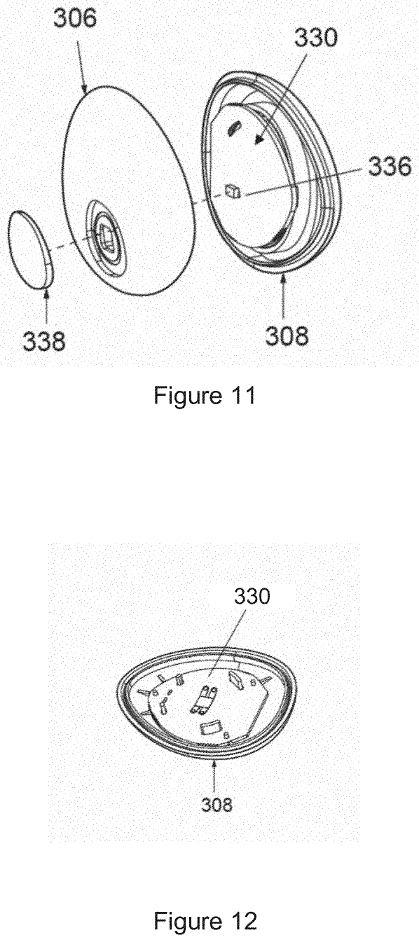

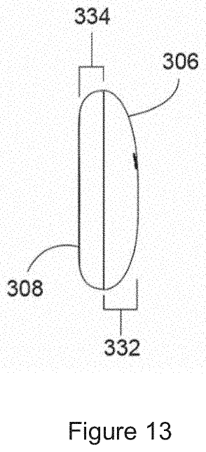

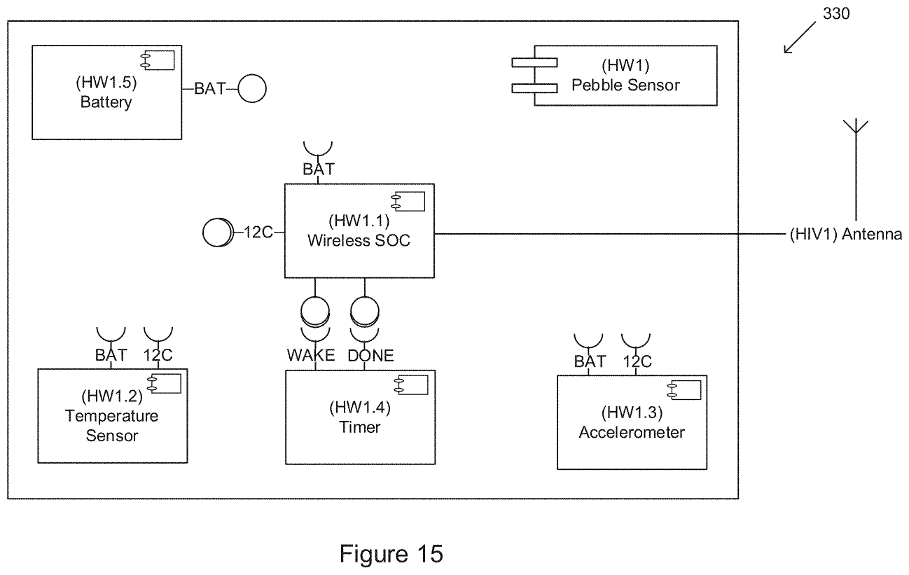

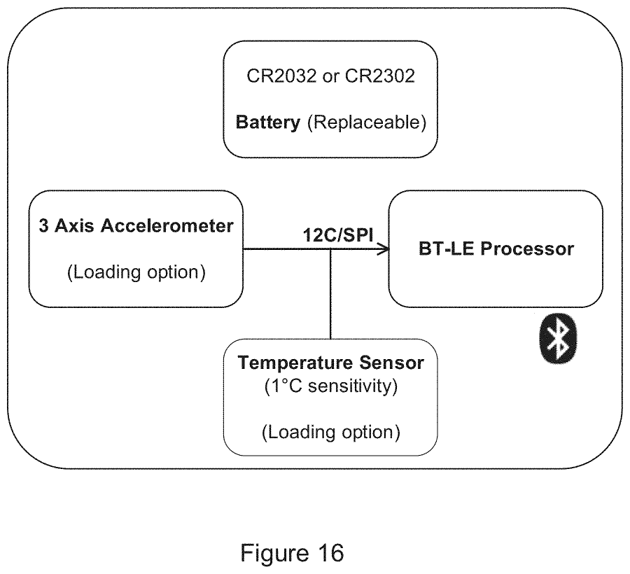

As shown in FIGS. 4, 13, 14A and 14B, the rigid sensor device 300 includes a housing 302 that houses an electronic system 330 of the rigid sensor device 300. The housing 302 is rigid, formed of at least one rigid material. The electronic system 330 includes electronic sensor components that detect the temperature and/or the motion, and generate respective electronic signals representing measurements of the temperature and/or the motion. As shown in FIG. 15, the electronic system 330 includes: an internal power source in the form of a battery HW1.5 (which is held in a battery holder of the rigid sensor device 300) that provides a power line BAT in the electronic system 330; a temperature sensor module HW1.2 configured to sense the temperature; a timer module HW1.4 configured to generate a WAKE signal; an accelerometer module HW1.3 configured to measure the acceleration of the rigid sensor device 300; a wireless module HW1.1, in the form of a system-on-a-chip (SOC), that receives signals from the temperature sensor module HW1.2 representing the measured temperature, and from the accelerometer module HW1.3 representing the measured the acceleration, and that transmits these signals to an antenna HIV1; and the antenna HIV1 for wireless communication, e.g., a commercially available Bluetooth antenna. The timer module HW1.4 sends WAKE signals to the wireless module HW1.1 to activate wireless module HW1.1 to transmit communication signals at selected times, thus providing a power-saving period in which the wireless module HW1.1 is not active and using battery power.

The electronic system 330 may include a gyroscope module configured to measure rotation of the rigid sensor device 300, including in 3 axes of rotation.

The rigid sensor device 300 has a flat shape, defined by a flat shape of the housing 302, and the elastic pocket 200 holds the rigid sensor device 300 flat against the sleeping garment 102. The rigid sensor device 300 has two opposite sides provided by its flat shape, an inner-facing side 332 and an outer-facing side 334, and the elastic pocket 200 holds the rigid sensor device 300 tightly to resist the rigid sensor device 300 flipping relative to an inner face 104 of the sleeping garment 102, i.e., such that the inner-facing side 332 remains facing an interior of the sleeping garment 102 (and thus facing toward the person) while the person wears the sleeping garment 102, including during natural movement of the person.

For reasons set out hereinafter, the at least one rigid material that forms the housing 302 includes: a thermal conductor 338 (also referred to as a “thermocouple” or “thermally conductive portion”) on its inner-facing side (which corresponds to the inner-facing side 332 of the rigid sensor device 300). Thus the rigid material includes: (i) a rigid thermally conductive material (for the thermal conductor), which can include metallic materials, e.g., aluminium, or another rigid material with high thermal conductivity; and (ii) a rigid material (for the rest of the housing 302), which can be include polymer materials, including thermoplastic polymers, e.g., a blend with acrylonitrile butadiene styrene (ABS) and polycarbonates, or another rigid material with low thermal conductivity.

The materials forming the housing 302 are:
a. food safe, in case the child puts the rigid sensor device 300 in or to its mouth;
b. fire retardant, in case the apparatus 100 is overheated, e.g., in a clothes dryer, or by being too close to a space heater;
c. shatter resistant, in case the apparatus 100 is struck hard, e.g., dropped or stepped on; and
d. detergent resistant, so the apparatus 100 can be washed with other clothes without harming the rigid sensor device 300.

The housing 302 is water resistant, to a rating of IP67, so the apparatus 100 can be washed with other clothes, or otherwise made wet, without harming the electronic system 330. The housing 302 is formed of water-resistant materials (metallic materials and polymer materials), and includes a water-resistant seal and a water-resistant weld.

The housing 302 can include a housing body formed in at least two portions: an inner-facing portion 306 (also referred to as a “front housing”) on the inner-facing side 332 and an outer-facing portion 308 (also referred to as a “back housing”) on the outer-facing side 334. These two portions 306,308 may each be formed with an open geometry, i.e., including no enclosed spaces to facilitate injection moulding and then assembled to form the housing 302. These two portions of the housing body are welded together along their join 304 to form the water-resistant weld, e.g., by ultrasonic welding.

To improve water resistance, the housing 302 includes two water-resistant seals 350, 352 around a battery door 310 in the rigid sensor device 300. The battery door 310 includes a male bayonet fitting that engages with a cooperative female bayonet receiver in the housing body of the housing 302. The battery door 310 is rotated into the housing body, thereby compressing the water-resistant seals 350, 352 (e.g., O-rings) between the battery door 310 and the housing body to improve water resistance of the housing 302.

The rigid sensor device 300 is configured to detect the temperature of the person by detecting the temperature inside the sleeping garment 102 while the person is wearing the sleeping garment 102. The rigid sensor device 300 includes one or more electronic temperature sensors 336 that detect the temperature by electronically monitoring a temperature of the thermal conductor 338 of the rigid sensor device 300. The thermal conductor 338 extends from the electronic temperature sensors 336 towards the inner-facing side 332 of the rigid sensor device 300 to be more sensitive to temperatures inside the sleeping garment 102 rather than temperatures outside the sleeping garment 102. Thus the housing 302 includes: the thermal conductor 338 in its inner-facing portion 306 (which corresponds to the inner-facing side 332 of the rigid sensor device 300). The thermal conductor 338 includes a thermocouple face 312 that forms a fraction of the area of the inner-facing side 332 of the housing 302: this fraction can be 100% of the area (i.e., the entire inner-facing side of the housing 302 can be formed by the thermocouple face 312), or less than 100%, including sufficient surface area to allow rapid thermal communication between the air in the sleeping garment 102 and the electronic temperature sensors 336. In an example, the fraction is between 2% and 20%, e.g., about 6.8%, and the area of the thermocouple face 312 is between 50 mm² and 200 mm², e.g., about 116 mm² compared to the area of the inner-facing side 332 of the housing 302 which can be between 1000 mm² and 4000 mm², e.g, 1705 mm². The thermocouple face 312 is held facing the interior of the sleeping garment 102, even during natural movement of the person wearing the sleeping garment 102, because the elastic pocket 200 holds the rigid sensor device 300 flat against the sleeping garment 102, i.e., relative to an inner face of the sleeping garment.

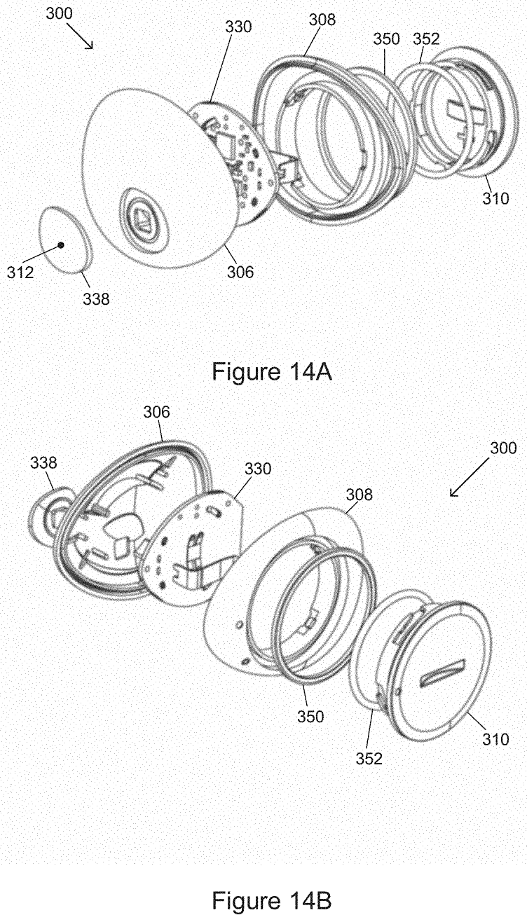

Detection of the motion of the rigid sensor device 300 includes detection of a flip of the sleeping garment 102, i.e., detecting that the person wearing the sleeping garment 102 has flipped or rolled from its back onto its front. The rigid sensor device 300 includes one or more electronic direction sensors (which can include accelerometers and/or gyroscopes, which can be the accelerometer module HW1.3) that detect the flip by electronically monitoring for a change in orientation of the rigid sensor device 300. The term “flip” refers to rotation of the rigid sensor device 300 or the person from its front to its back, either quickly or slowly. For the rigid sensor device 300, the flip involves the two opposite sides swapping vertical orientation. As shown in FIG. 15, the electronic direction sensors can include the accelerometer module HW1.3, which can be a 3-axis accelerometer module, in communication with a transmitter, e.g., in a Bluetooth low-energy (BT-LE) processor, via a communications bus, e.g., an I2C or SPI bus. As shown in FIG. 14, the one or more electronic direction sensors can be the accelerometer module HW1.3 connected to the I2C bus and the power line BAT.

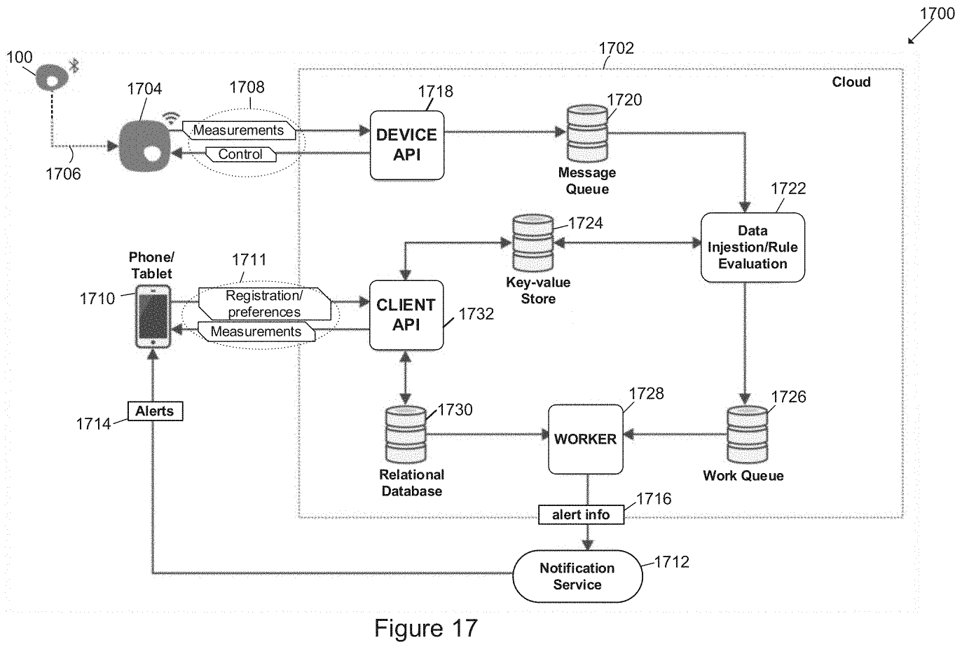

As shown in FIG. 17, the apparatus 100 is part of a system 1700 for monitoring the person, including while sleeping. This system 1700 includes the electronic system 330, and this system 1700 performs a flip-detection method described hereinafter as part of a method for monitoring the person, including while sleeping.

The housing 302 includes:
a. the housing body, including the inner-facing portion 306 and the outer-facing portion 308, e.g., formed of a plastic material by injection moulding;
b. the battery door 310 that bayonet fits into the housing body;
c. the water-resistant seals 350, 352 that fit between the housing body and the battery door 310; and
d. the thermal conductor 338, e.g., formed of the metallic material, and glued into the housing 302 to form a water-tight seal.

The elastic pocket 200 includes an elastic material that stretches when the rigid sensor device 300 is inside the elastic pocket 200 to hold the rigid sensor device 300 tightly.

The elastic pocket 200 includes an openable closure 206 that: (i) when open, allows the rigid sensor device 300 to enter the elastic pocket 200; and (ii) when closed, holds the rigid sensor device 300 in the elastic pocket 200. The openable closure 206 is configured to be manually operated (i.e., opened and closed) by an adult while resisting manual operation by a child.

The openable closure 206 includes an elastic throat 208 through which the rigid sensor device 300 is pushed by the adult to insert the rigid sensor device 300 into the elastic pocket 200, and to remove the rigid sensor device 300 from the elastic pocket 200: this insertion and removal requires sufficient force to resist manual operation by a child, e.g., the force may be at least 5 Newtons (N), and no more than 15 Newtons (N). This insertion/removal force is defined by: (i) the elasticity of the material forming the elastic throat 208, (ii) the relative sizes of the elastic throat 208's width 210 and rigid sensor device 300's width 314, and (iii) a shape of the housing 302 where it presses against the elastic throat 208, i.e., shapes of camming surfaces provided by a smoothly increasing section 322 and a maximum-width section 324 of the housing 302). The housing 302 has a surrounding edge between its two opposite sides (i.e., along the join 304), and the surrounding edge defines, in order: (i) a narrow-end section 320 with a width less than the elastic throat 208 width to allow easy insertion into the elastic throat 208; (ii) the smoothly increasing section 322 (i.e., a tapered section) that presses increasingly (i.e., gradually and monotonically) against the elastic throat 208 as the rigid sensor device 300 is inserted into the elastic throat 208; (iii) the maximum-width section 324 (in a direction perpendicular to a long axis of the rigid sensor device 300) with the width 314 more than the elastic throat 208 where the sufficient manual force is required to force the rigid sensor device 300 further into the elastic throat 208; and (iv) a tail-end section 326, with a width less than the elastic throat 208 width, that extends sufficiently beyond the maximum section to allow the user to push the rigid sensor device 300 completely into and/or though the elastic throat 208. As the rigid sensor device 300 is substantially flat, the forces between the elastic throat 208 and the rigid sensor device 300 are defined substantially by the shape of the surrounding edge (along the join 304). The user manually directs the narrow-end section 320 into the elastic throat 208, and forces the rigid sensor device 300 through the elastic throat 208 into its natural or correct operational position in the elastic pocket 200. Once inside the elastic pocket 200, the user removes the rigid sensor device 300 by manually directing the narrow-end section 320 into the elastic throat 208, and forcing the rigid sensor device 300 back through the elastic throat 208 and out of the elastic pocket 200.

The rigid sensor device 300 is longer than it is wide (i.e., an elongated device, with a length 316 along the long axis greater than the width 314 perpendicular to the long axis), and the elastic pocket 200 may include an elastic pouch 212 (separate from, and at the end of, the elastic throat 208—and formed of the elastic material described hereinbefore) that is also longer than it is wide (i.e., an elongated pouch, with a length 216 greater than a width 214), and the natural elasticity of the elastic pocket 200 may allow (and encourage) the rigid sensor device 300 to be manually rotated so a longer dimension of the rigid sensor device 300 aligns with a longer dimension (along the long axis) of the elastic pouch 212. The rigid sensor device 300 and the elastic pouch 212 are both flat (thus defining a main axis through a main plane of the rigid sensor device 300 and the elastic pouch 212), so the rigid sensor device 300 is held flat in the elastic pocket 200 as described hereinbefore (thus the rigid sensor device 300 tends not to flip along its main axis relative to the elastic pocket 200); however, in addition to being flat (to resist flipping), the rigid sensor device 300 and the elastic pouch 212 can also be elongated to resist “flat rotation”, i.e., rotation of the rigid sensor device 300 in the elastic pocket 200 around its main axis. The elastic pouch 212's longer dimension (along its length 216) is perpendicular to the insertion direction of the elastic throat 208, thus the rigid sensor device 300 is naturally retained in the elastic pocket 200 with its longer dimension (along its length 316) facing the elastic throat 208's entry (i.e., the longer dimension is perpendicular to the insertion direction of the elastic throat 208), thus the elastic throat 208 resists any movement of the rigid sensor device 300 into the elastic throat 208 when the rigid sensor device 300 is in its natural or correct operational position in the elastic pocket 200.

The openable closure 206 includes an openable flap 218 that, when closed, blocks access to the openable closure 206 from outside the elastic pocket 200. The flap 218 closes over the elastic throat 208 so the rigid sensor device 300 cannot be forced through the elastic throat 208. The flap 218 includes an elastic material so it can be manually held open to force the rigid sensor device 300 through the openable closure 206, i.e., due to its natural resilience (or elasticity); however, the flap 218 is arranged to naturally fall back to its closed arrangement when not being held open. The flap 218 can be attached along a top edge 220 of the elastic throat 208, and down both sides 222 of the elastic throat 208, e.g., between 20 mm and 35 mm, at least as far as, but not extending onto, the thermocouple face 312.

The openable closure 206 may include other fastening mechanisms that retain the rigid sensor device 300 in the elastic pocket 200, and that resist manual opening by a child.

The elastic pocket 200 includes an inner-facing fabric (or material), corresponding to the inner-facing side 332 of the rigid sensor device 300, that is relatively thermally permissive (relative to a body fabric of the sleeping garment 102, i.e., the fabric that forms the body of the sleeping garment 102 and keeps the person warm) to mitigate thermal differences between the air inside the sleeping garment 102 (adjacent the elastic pocket 200) and the inner-facing side of the rigid sensor device 300 (inside the elastic pocket 200) where the thermocouple face 312 is formed. In other words, the thermally permissive fabric mitigates the thermal difference between air inside the sleeping garment and the inner-facing side of the rigid sensor device relative to a thermal differences between air inside the sleeping garment and air outside the sleeping garment. The inner-facing fabric is configured to be thermally permissive by: (i) pressing tight to the thermocouple face 312 due to the natural elasticity of the elastic pocket 200 (thus minimising any air gap between the air inside the sleeping garment 102 and the thermocouple face 312); (ii) including an open mesh fabric 202 (e.g., polyester mesh) to conduct heat well between the air inside the sleeping garment 102 and the thermocouple face 312.

The elastic pocket 200 includes location markers 224 on the inner-facing fabric that are arranged to align with inner-facing features of the rigid sensor device 300 when the rigid sensor device 300 is in its natural and correct operational location in the elastic pocket 200. The location markers 224 can include a different fabric from the thermally permissive inner-facing fabric, i.e., a fabric that is less thermally permissive, and that is visually distinguishable, which may be a layer on the thermally permissive inner-facing fabric, e.g., a polymer strip, e.g., a vinyl print. The inner-facing features of the rigid sensor device 300 may be the edge or join 304, and a boundary 328 (or dividing line) between the thermocouple face 312 (e.g., in aluminium) and the housing 302 (e.g., in polymer), i.e., an outline of the thermocouple face 312. The location markers 224 may include a matching outline to the boundary 228 of the thermocouple face 312, and a matching outline to the edge along the join 304. The inner-facing fabric can be largely optically transparent so the rigid sensor device 300 can be seen inside the elastic pocket 200 to assist a user (generally an adult) who is manually arranging the rigid sensor device 300 into the correct operational location. The thermocouple face 312 differs optically from the housing, e.g., being formed of the metallic material instead of the polymer materials, so the user can see that the thermocouple face 312 through the inner-facing fabric, and thus see whether the rigid sensor device 300 is in the correct operational location with thermocouple face 312 facing inwards.

The apparatus 100 may include: a plurality of the rigid sensor device 300s and a corresponding plurality of the elastic pocket 200s (including in a single shared sleeping garment 102), and each of the plurality of rigid sensor device 300s may include respective markings to indicate which of the rigid sensor device 300s is generating which of the electronic signals representing measurements of the temperature and/or the motion. The respective markings may include respective different coloured housings.

The elastic pocket 200 is attached to the sleeping garment 102, inside the sleeping garment 102, at a location adjacent the person's torso (including the person's stomach or chest) while the person is wearing the garment. The elastic pocket 200 may include a fabric backing piece 204 that is attached to the sleeping garment 102 (by sewing), or the elastic pocket 200 may be sewn into the sleeping garment 102 directly so an inner fabric of the body of the sleeping garment 102 (i.e., a portion of the body fabric) forms the backing piece.

A method for making the apparatus 100 includes the following steps:
a. providing the sleeping garment 102;
b. attaching the elastic pocket 200 to the sleeping bag garment for tightly holding the rigid sensor device 300 inside the sleeping garment 102 while the person is wearing the sleeping garment 102; and
c. configuring the rigid sensor device 300 for detecting the temperature and/or the motion of the sleeping garment 102 (and thus of the person).

In more detail, the method for making (i.e., manufacturing or assembling) the apparatus 100 includes the following steps:
a. assembling electronic sensor components onto a printed circuit board assembly (PCBA) of the electronic system 330;
b. forming the two portions 306, 308 of the housing body and the battery door 310, e.g., by injection moulding;
c. forming the thermal conductor 338 (including the thermocouple face 312), e.g., by casting;
d. assembling the thermal conductor 338 into the front housing 306, e.g., by glue;
e. assembling the PCBA into the front housing 306 or the back housing 308;
f. welding the water-resistant join 304 between the two portions 306, 308 of the housing body;
g. attaching the battery HW1.5 (e.g., of type CR2032 or CR2302) to the electronic sensor components by inserting the battery HW1.5 into the battery holder (in the housing 302 or the battery door 310), and rotating the battery door 310 into the housing body against the water-resistant seals 350, 352;
h. sewing/bonding the elastic pocket 200, including forming the shape of the elastic pocket 200, and the openable closure 206 (which may include the elastic throat 208), and the flap 218;
i. sewing/bonding the pocket 200 into the garment 102;
j. manually inserting, by an adult, the rigid sensor device 300 into the elastic pocket 200 (through the openable closure 206), including (as appropriate) manually holding the flap 218 open, pushing the rigid sensor device 300 through the elastic throat 208 in a first orientation (i.e., with the long axis of the elongated rigid sensor device 300 aligned with the elastic throat 208 insertion direction), and rotating the rigid sensor device 300 when in the elastic pocket 200 from the first orientation to a second orientation (that is perpendicular to the first orientation, i.e., to have the long axis of the rigid sensor device 300 aligned perpendicular to the elastic throat 208 insertion direction, and parallel to the long axis of the elongated elastic pocket 200).

The method for monitoring a person, including while sleeping, includes a method of operation of the apparatus 100, which includes the steps of:
a. holding the rigid sensor device 300, by the elastic pocket 200, tightly inside the sleeping garment 102 to resist flipping of the rigid sensor device 300 relative to the sleeping garment 102; and
b. electronically monitoring the temperature and/or the motion of the rigid sensor device 300 using the electronic sensor components while the person wearing the sleeping garment 102.

The method for monitoring the person includes the flip-detection method mentioned hereinbefore. The flip-detection method includes:
a. electronically monitoring for a change in orientation of the rigid sensor device 300 by the one or more electronic direction sensors measuring an axis, e.g., the Z-axis, which is the vertical axis;
b. the system detecting the change in orientation of the rigid sensor device 300 when the vertical axis (e.g., the vertical one of the accelerometer axes or the vertical one of the gyroscope axes) changes direction more than a selected threshold (referred to as a “flip threshold”), which can be 50%, 60%, 70%, 75%, 80%, 80% or 90% towards the opposite direction; and
c. the system generating a flip alert or alarm signal when the direction change (or “flip”) is detected.

The Z-axis may be represented by value +15,000 to −15,000. The electronic system 330 may sample the electronic direction sensors every 1 second. If within 1 sample (1 second), the Z value changes by an amount of 12,000 or more, to the opposite direction, the system raises the a flip alert or alarm signal (referred to as a “flip-over event flag”), and sends it to a remote computing system 1702 as shown in FIG. 17, which can be a cloud-computing system, to send an alert to a local or remote alarm system to warn a carer of the flip event, and/or to wake the person. The comparison of the Z-axis value to the threshold (e.g., 12,000) can be performed by the electronic system 330, or can be performed in the remote computing system 1702.

As shown in FIG. 17, the system 1700 for monitoring the person, including while sleeping, includes:
a. the apparatus 100, which has the electronic system 330 that generates the respective electronic signals representing measurements of the temperature and/or the motion, and that transmits the signals from the antenna HIV1 using a wireless protocol, e.g., Bluetooth or low-energy Bluetooth;
b. the remote computing system 1702, which is remote from the apparatus 100, e.g., the remote computing apparatus 1702 can be a portion of a commercial computing cloud;
c. a bridge 1704 that provides an electronic communications bridge between the apparatus 100 and the remote computing system 1702 by receiving the signals from the apparatus 100 over a wireless link 1706 provided by the antenna HIV1 and a corresponding antenna in the bridge 1704, and a remote communications connection 1708 with a remote computing system 1702 to send the measurements from the electronic sensor components to the remote computing system 1702, and to receive control signals from the remote computing system 1702;
d. an end user device 1710, e.g., a commercially available smart phone, tablet or personal computer that communicates with the remote computing system 1702 using a user communications link 1711, e.g., an encrypted and authenticated communications link, for the user device 1710 to receive the measurements, and to send registration and preference parameters to the remote computing system; and
e. a notification system 1712, such as a short-message-service (SMS) system and/or an automated text-to-speech system, that connects to the remote computing system 1702 to receive alert information, and that generates and sends alerts 1714 to the user device 1710 under the control of the alert information 1716.

The notification system 1712 can include a text-to-speech module to convert written information in the alert information 1716, including the measurement values and threshold values that led to the alert being triggered, so the alerts 1716 can include voice calls as well as messages. The alerts 1716 may be referred to as “push alerts” because they are pushed to the user device 1710 as soon as the alert conditions are satisfied.

The remote computing system 1702 includes:
a. a device application programming interface (API) 1718, which communicates with the link 1708, and transmits requests to a message queue 1720;
b. the message queue 1720, which queues the requests for transmission to a data ingestion/rule evaluation module 1722;
c. the data ingestion/rule evaluation module 1722, which communicates with a key-value store 1724 and communicates with a work queue 1726;
d. the key-value store 1724 that stores data representing the measurements, and stores configuration parameters from the user device 1710;
e. the work queue 1726, which queues tasks for a work module 1728 to generate the alert information 1716;
f. the work module 1728, which generates the alert information 1716 based on alert conditions determined by the data ingestion/rule evaluation module 1722 received via the work queue 1726, and communications parameters stored in a communications database 1730;
g. the communications database 1730, which is populated with data from the user device 1710; and
h. a client API 1732 that receives the communications attributes from the device 1710, and sends the measurements to the user device 1710, e.g., the stored measurements from the key-value store 1724.

In the flip-detection method described hereinbefore, the apparatus 100 measures the orientation of the rigid sensor device 300 by way of the electronic system 330 therein. The electronic system 330 may itself determine that the orientation is changed more than the selected flip threshold, and may itself generate the flip alert alarm signal, e.g., a wireless signal sent over the link 1706 to the bridge 1704, which can then generate audible and/or visual alarms, and can control the remote computing system 1702 to generate the alert information 1716 to generate the alert 1714 for the user device 1710. Alternatively or additionally, the electronic system 330 may send the measurement values, i.e., obtained directly from the electronic sensors, over the link 1706 to the bridge 1704, which itself can determine whether the flip threshold has been met/exceeded, and itself generate the flip alert or alarm signal. Alternatively or additionally, the bridge 1704 can send the raw measurement values to the remote computing system 1702, which can determine in the rule ingestion/rule evaluation module 1722 whether the flip threshold has been met. Alternatively or additionally, the bridge 1704 can determine whether the flip threshold has been met, and then control the remote computing system 1702 to generate the alert information 1716 in consequence.

The data ingestion/rule evaluation module 1722 is controlled by different alert rules, which are stored in the key-value store 1724, which are selected to be active, i.e., to be applied by the data ingestion/rule evaluation module 1722, by the user device 1710 selecting which of the rules are to be applied based on the identifying parameters of the rules. One of these rules is a temperature alert rule, which causes the data ingestion/rule evaluation module 1722 to activate an alert request when the temperature measurements it receives, via the bridge 1704, are above or below a selected maximum/minimum temperature threshold selected by the user device 1710, including for more than a selected minimum time period (which can be selected by the user device 1710). The alert rules can include a rollover alert (as known as the flip alert), when a flip is detected as described hereinbefore. The alert rules can also include an off-line alert which is determined by the data ingestion/rule evaluation module 1722 when no measurements are received by the data-ingestion/rule evaluation module 1722 for more than a selected wait-time period, which is selected by the user device 1710, and stored in the key-value store 1724.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. An apparatus for monitoring a person, including while sleeping, the apparatus including:
a. a garment for a person to wear while sleeping;
b. a rigid sensor device configured to detect a temperature and/or a motion of the rigid sensor device while the person is wearing the garment; and
c. an elastic pocket configured to hold the rigid sensor device tightly inside the garment during the detection of the temperature and/or the motion.

2. The apparatus of claim 1, the rigid sensor device including:
a housing; and
an electronic system in the housing.

3. The apparatus of claim 2, wherein the electronic system includes electronic sensor components configured to detect the temperature and/or the motion of the garment, including at least one electronic direction sensor, wherein the rigid sensor device is configured to detect the motion of the garment by detecting a flip of the garment using the at least one electronic direction sensor.

4. The apparatus of claim 3, wherein the at least one electronic direction sensor includes at least one of an accelerometer and a gyroscope.

5. The apparatus of claim 2, wherein the electronic system includes any one or more of:
an internal power source that provides power in the electronic system;
a temperature sensor module configured to sense the temperature;
a timer module configured to generate a WAKE signal;
an accelerometer module configured to measure an acceleration of the rigid sensor device;
a gyroscope module configured to measure rotation of the rigid sensor device;
a wireless module configured to receive signals from the temperature sensor module representing the temperature, and/or from the accelerometer module representing the measured acceleration; and
an antenna configured to communicate wirelessly.

6. The apparatus of claim 2, wherein the housing is formed of at least one rigid material including a thermal conductor or a thermally conductive portion, and the rigid sensor device is configured to detect the temperature of the garment by electronically monitoring a temperature of the thermal conductor or thermally conductive portion.

7. The apparatus of claim 1, wherein the elastic pocket includes an openable closure with an elastic throat through which the rigid sensor device can be inserted and removed from the elastic pocket, wherein the rigid sensor device has a maximum width section, in a direction perpendicular to a long axis of the rigid sensor device, with a width greater than the elastic throat, such that the elastic throat resists the insertion and the removal of the rigid sensor device into and from the elastic pocket,
optionally wherein the insertion and the removal of the rigid sensor device requires a force between 5 and 15 N.

8. The apparatus of claim 1, wherein the elastic pocket includes an elastic pouch,
wherein the rigid sensor device and the elastic pouch are both flat so the rigid sensor device is held flat in the elastic pocket, and
wherein the elastic pouch is longer than it is wide so a longer dimension of the rigid sensor device can align with a longer dimension of the elastic pouch to resist flat rotation of the rigid sensor device.

9. The apparatus of claim 1, wherein the elastic pocket includes an inner-facing fabric, corresponding to an inner-facing side of the rigid sensor device, that is thermally permissive relative to a body fabric of the garment to mitigate thermal differences between air inside the garment and the inner-facing side of the rigid sensor device.

10. The apparatus of claim 1, wherein the rigid sensor device includes a housing body, a battery door, a water-resistant join and/or a water-resistant seal.

11. A method for making an apparatus, the method including:
providing a garment;
attaching an elastic pocket to the garment for tightly holding a rigid sensor device inside the garment while a person is wearing the garment; and
configuring the rigid sensor device to detect a temperature and/or a motion of the rigid sensor device.

12. The method of claim 11, including any one or more of:
assembling electronic sensor components to form an electronic system of the rigid sensor device;
forming a housing body of the rigid sensor device, including two portions and a battery door, and optionally forming the housing body with a water-resistant join and/or a water-resistant seal;
forming a thermal conductor of the rigid sensor device;
inserting a battery into a battery holder of the rigid sensor device; and
sewing and/or bonding the elastic pocket into the garment.

13. A method for monitoring a person, including while sleeping, the method including:
holding a rigid sensor device, by an elastic pocket, tightly inside a garment to resist movement of the rigid sensor device relative to the garment; and
electronically monitoring a temperature and/or a motion of the rigid sensor device while a person is wearing the garment.

14. The method of claim 13, including:
housing an electronic system for the electrical monitoring in a water-resistant housing.

15. The method of claim 13, including detecting a flip of the garment by:
electronically monitoring for a change in orientation of the rigid sensor device by one or more electronic direction sensors measuring an axis;
detecting the change in orientation of the rigid sensor device when the axis changes direction more than a selected threshold (“flip threshold”); and
generating a flip alert or alarm signal when the direction change (or “flip”) is detected.

16. The method of claim 13, including any one or more of:
powering the rigid sensor device with an internal power source;
sensing the temperature using a temperature sensor module of the rigid sensor device;
generating a WAKE signal to transmit communication signals;
measuring the motion using an accelerometer module and/or a gyroscope module of the rigid sensor device;
transmitting wireless communication signals using a wireless module and an antenna of the rigid sensor device; and
electronically monitoring a temperature of a thermal conductor or a thermally conductive portion of the rigid sensor device.

17. The method of claim 13, including any one or more of:
manually inserting the rigid sensor device into the elastic pocket, optionally including holding open a flap of the elastic pocket; and
manually removing the rigid sensor device from the elastic pocket.

18. The method of claim 17, wherein the manually inserting includes:
pushing the rigid sensor device through an elastic throat of the elastic pocket in a first orientation, optionally with a force between 5 and 15 N; and
rotating the rigid sensor device when in the elastic pocket from the first orientation to a second orientation that is perpendicular to the first orientation.

19. The method of claim 13, including:
the elastic pocket holding the rigid sensor device flat relative to an inner face of the garment; and
the elastic pocket resisting rotation of the rigid sensor device in the elastic pocket.

20. The method of claim 13, including:
mitigating a thermal difference between air inside the garment and an inner-facing side of the rigid sensor device relative to a thermal differences between air inside the garment and air outside the garment.
